(19) United States
(12) Patent Application Publication
(10) Pub. No.: US 2021/0322521 A1
(43) Pub. Date: Oct. 21, 2021

(54) COMPOSITIONS AND METHODS FOR TREATING CORNEAL ENDOTHELIUM

(71) Applicant: D&D Biopharmaceuticals, Inc., Vienna, VA (US)

(72) Inventors: Bruce H. DeWOOLFSON, Vienna, VA (US); Dale P. DeVore, Chelmsford, MA (US); Richard A. Eiferman, Louisville, KY (US); Debra Skelnik, Cambridge, MN (US)

(73) Assignee: D&D Biopharmaceuticals, Inc., Vienna, VA (US)

(21) Appl. No.: 16/852,969

(22) Filed: Apr. 20, 2020

(51) Int. Cl. A61K 38/39; A61K 9/06; A61K 9/08; A61K 9/70; A61K 9/00; A61K 47/36; A61P 41/00

(52) U.S. Cl. CPC A61K 38/39; A61K 9/06; A61K 9/08; A61P 41/00; A61K 9/0048; A61K 47/36; A61K 9/7007

(57) ABSTRACT

Compositions of proteoglycans and core proteins thereof and related methods of treating subjects, e.g., patients, are described. The method may include applying a composition to an eye of a subject before, during, and/or after an intraocular medical procedure. The composition may comprise a proteoglycan or core protein thereof, such as decorin proteoglycan or decorin core protein, and a pharmaceutically-acceptable salt. The composition may be in the form of an aqueous solution, viscoelastic gel, or viscoelastic film, for example. Further methods are described that include treating donor corneal tissue with a composition comprising a proteoglycan or core protein thereof. The treated donor corneal tissue may be used as or in a corneal graft, such as during endothelial keratoplasty.

### TECHNICAL FIELD

The present disclosure relates to methods for treating corneal tissue. For example, the present disclosure includes methods for maintaining corneal endothelial cell anatomy, function, and/or structure before, during, and/or after a medical procedure.

### BACKGROUND

The structure of the eye includes an anterior segment that includes the outermost components (cornea, iris, and lens) and an inner posterior segment. Disorders of the anterior segment in particular can impair vision, and may cause damage and/or deterioration in the lens or cornea. One concern is loss or damage to endothelial cells. The cornea includes five basic layers including a thin outer epithelium, Bowman's membrane, the thick stroma, Descemet's membrane, and a thinner endothelium. That is, the corneal endothelium forms the inner surface of the cornea, facing the anterior chamber. Corneal endothelial cells are involved in fluid and nutrient diffusion across the cornea.

One common eye disorder is cataracts. A cataract is a clouding of the lens of the eye that affects vision. Most cataracts are related to aging. The risk of cataracts increases with each decade of life starting around age 40. By age 75, about half of Americans are expected to have a cataract. More than 3.8 million cataract surgeries are performed each year in the U.S., and it is estimated that globally there are more than 100 million eyes with cataracts causing a visual acuity less than 20/60.

Today, cataracts can be treated through a procedure that removes the cloudy lens and replacement with a “polymeric” intraocular lens. In order to accomplish this replacement, the native lens structure must first be removed. The standard procedure used today is phacoemulsification, which was developed by Dr. Charles Kelman in 1967. Phacoemulsification involves the application of ultrasound pulses to rupture the cataract lens structure into small fragments that are removed by aspiration. During this process, the anterior chamber of the eye is typically flooded with a physiological solution, such as balanced salt solution (e.g., saline solution) or filled with ophthalmic viscoelastic devices (OVDs) to provide some protection to the corneal endothelium during the phacoemulsification procedure.

However, even with the OVD protection, there is typically loss of corneal endothelial cells. Anterior and posterior segment surgery conducted using phacoemulsification or laser assisted surgery typically results in damaging effects on corneal endothelium. Endothelial cells are generally nonreplicative, and loss is only compensated by migration, enlargement, and increasing heterogeneity of cells. Loss of endothelial cell function can lead to increased corneal thickness and decreased corneal transparency due to compromised pump function and increased stromal hydration, and can result in development of endothelium-epithelium dystrophy and sharp loss of eyesight. While it has been suggested that OVDs provide some protection, such protection is minimal.

Other eye disorders are associated with loss or damage to endothelial cells. Fuchs endothelial cell dystrophy refers to swelling of the cornea due to loss of endothelial cells and proper regulation of fluid. Vision worsens with increasing corneal endothelial cell death over time. Pseudophakic corneal edema refers to irreversible corneal edema and endothelial cell damage due to cataract removal, and often following placement of an intraocular lens. Treatment for these conditions can include transplantation of donor corneas. These donor materials are typically stored in an eye bank, in a preservation medium prior to use. However, reduction in endothelial cell density can occur over time, which may lead to failures when used to replace a defective endothelium in a patient.

### SUMMARY

The present disclosure includes compositions and methods for treating patients, e.g., humans and non-human mammals. The compositions and methods herein may be useful in preserving and maintaining endothelial cell density, and/or preserving, maintaining, and/or increasing cell structure. For example, the methods herein include one or more direct applications of a composition to the endothelium before, during, and/or after a medical procedure affecting native corneal tissues e.g., an intraocular medical procedure, such as cataract surgery. The methods herein further include treating corneal tissues prior to implantation in a subject, e.g., as a graft or other material to supplement or replace native corneal tissues. Application of a composition comprising one or more proteoglycans and/or proteoglycan core proteins, such as decorin proteoglycan and/or decorin core protein, may preserve endothelial cell density and/or results in an increase in hexagonal cell structure.

For example, the present disclosure includes methods of treating a subject (e.g., a patient), wherein the method may comprise applying a composition to an eye of the subject during an intraocular medical procedure, and wherein the composition comprises a proteoglycan or proteoglycan core protein. The proteoglycan or core protein thereof may comprise, for example, decorin, biglycan, keratocan, lumican, mimican, fibromodulin, epiphycan, or a core protein thereof. Optionally, the composition may further comprise a pharmaceutically-acceptable salt. Exemplary salts include sodium chloride, potassium chloride, calcium chloride, magnesium chloride, sodium acetate, sodium citrate, and combinations thereof. Additionally or alternatively, the composition may comprise hyaluronic acid, sodium hyaluronate, chondroitin sulfate, dermatan sulfate, heparin sulfate, keratin sulfate, hydroxyl propylmethylcellulose, recombinant human collagen, or a combination thereof. The composition may comprise a solution, gel, or film, such as an aqueous solution, a viscoelastic gel, or a viscoelastic film. The composition optionally may comprise cross-linked hyaluronic acid or sodium hyaluronate, chondroitin sulfate, dermatin sulfate, heparin sulfate, keratin sulfate, hydroxylpropylmethylcellulose, or a combination thereof.

In at least one example, the concentration of the proteoglycan or proteoglycan core protein in the composition ranges from about 0.001 mg/mL to about 10 mg/mL. The composition may have a pH ranging from 6.0 to 8.0. The subject may be a human or non-human mammal, for example. According to some aspects of the present disclosure, the composition is applied to endothelial tissue. The intraocular medical procedure may be or include cataract surgery, e.g., phacoemulsification or high frequency pulsed vacuum cataract surgery. In some examples, applying the composition maintains endothelial mean cell density and/or increases or maintains hexagonal cell structure. An exemplary volume of the composition applied to the eye may range from about 0.050 mL to about 2.0 mL, e.g., about 1.0 mL. The composition may be applied to one or more of the following: the eye, the cornea, endothelial tissue, anterior chamber segment tissue, the posterior chamber of the eye, the retina, and/or the epithelium. The composition may be applied to the anterior chamber of the eye, for instance. The composition may directly contact the native corneal endothelium of the eye.

According to some aspects of the present disclosure, two or more different compositions comprising a proteoglycan or core protein thereof may be applied to the subject. For example, the composition may be a first composition, the method further comprising applying a second composition different from the first composition to the subject's eye, wherein the second composition comprises decorin proteoglycan or decorin core protein, or another proteoglycan or core protein thereof. In at least one example, the second composition is applied directly to the surface of the cornea of the eye after completing the intraocular medical procedure. Additionally or alternatively, the second composition may be applied directly to the surface of the cornea of the eye before completing the intraocular medical procedure.

The present disclosure also includes a method of treating a subject, comprising applying a composition to endothelial tissue of the subject during an intraocular medical procedure, wherein the composition comprises a proteoglycan or proteoglycan core protein, the composition being in the form of an aqueous solution, or a viscoelastic gel or viscoelastic film comprising, e.g., chemically derivatized and crosslinked collagen. The composition may comprise, e.g., decorin proteoglycan or decorin core protein, and optionally one or more of hyaluronic acid, sodium hyaluronate, chondroitin sulfate, dermatin sulfate, heparin sulfate, keratin sulfate, hydroxylpropylmethylcellulose, recombinant human collagen, or a combination thereof. In at least one example, the composition comprises cross-linked hyaluronic acid or sodium hyaluronate. In some examples, the intraocular medical procedure is phacoemulsification and or high frequency pulsed vacuum cataract surgery.

The present disclosure also includes a method of treating a subject, the method comprising applying a composition comprising a proteoglycan or core protein thereof (e.g., decorin proteoglycan or decorin core protein) to an eye of the subject during an intraocular medical procedure; wherein the composition is or comprises an aqueous solution, a viscoelastic gel, and/or a viscoelastic film. The intraocular medical procedure may treat cataracts, for example. The composition may be applied before and/or after phacoemulsification process, and/or before and/or after a high frequency pulsed vacuum process. In at least one example, the concentration of the proteoglycan or proteoglycan core protein in the composition ranges from about 0.001 mg/mL to about 10 mg/mL, and/or the total volume of the composition ranges from about 0.050 mL to about 2.0 mL, such as, e.g., about 1.0 mL.

Further aspects of the present disclosure include a method of preparing a corneal graft, the method comprising: treating donor corneal tissue with a composition comprising a proteoglycan or proteoglycan core protein, wherein the corneal graft includes the treated donor corneal tissue. For example, the proteoglycan or proteoglycan core protein may comprise decorin, biglycan, keratocan, lumican, mimican, fibromodulin, epiphycan, or a core protein thereof. Optionally, the composition may further comprise heparin, heparan sulfate, chondroitin sulfate, dermatan sulfate, heparin sulfate, keratan sulfate, hyaluronic acid, sodium hyaluronate, hydroxylpropylmethylcellulose, or a combination thereof. Additionally or alternatively, the composition may further comprise cross-linked hyaluronic acid, sodium hyaluronate, chondroitin sulfate, dermatin sulfate, heparin sulfate, keratin sulfate, hydroxylpropylmethylcellulose, or a combination thereof.

According to some aspects of the present disclosure, the composition further comprises an antibiotic agent (e.g., antibacterial agent), antioxidant, amino acid, cell nutrient, polysaccharide, organic molecule, biologic molecule, or combination thereof. In at least one example, the composition is, comprises, or serves as, a preservation medium.

The donor corneal tissue may be obtained from an eye bank, for example. According to some aspects of the present disclosure, the donor corneal tissue is treated for at least 10 minutes, e.g., about 10 minutes to about 30 days, or about 10 minutes to about 14 days.

The treated whole donor corneal tissue may enhance retention of the cell density of the cornea and/or may reduce endothelial cell loss as compared to untreated corneas. Additionally or alternatively, the treated donor corneal tissue may exhibit stability of, and/or an increase in, hexagonal cell structure as compared to the untreated donor corneal tissue. The treated whole donor corneal tissue may exhibit stability and/or greater corneal deturgescence as compared to untreated corneas.

The present disclosure further includes a method of preparing a corneal graft, the method comprising treating donor corneal tissue for at least 10 minutes, e.g., about 10 minutes up to 30 days, or about 10 minutes up to about 14 days, with a preservation medium comprising a proteoglycan or core protein thereof (e.g., decorin, biglycan, keratocan, lumican, mimican, fibromodulin, epiphycan, or a core protein thereof), wherein the corneal graft includes the treated donor corneal tissue. The treated donor corneal tissue may have a stable cell density and/or a stable hexagonal cell structure, and/or may exhibit higher corneal endothelial cell density and/or greater hexagonal cell structure as compared to a control (untreated) donor corneal tissue. In at least one example, the donor corneal tissue is treated for a period of time ranging from about 10 minutes to about 14 days, optionally at a temperature of about 2° C. to about 8° C., and/or treated for a period of time ranging from about 10 minutes to about 14 days at about 35° C.

The present disclosure also includes a method of treating a subject, the method comprising applying a corneal graft to an eye of the subject, wherein the corneal graft comprises donor corneal tissue treated with a composition comprising a proteoglycan or core protein thereof, e.g., decorin, biglycan, keratocan, lumican, mimican, fibromodulin, epiphycan, or a core protein thereof, wherein the corneal graft includes the treated donor corneal tissue. The treated donor corneal tissue may have a stable cell density and/or a stable hexagonal cell structure, and/or may exhibit higher corneal endothelial cell density and/or greater hexagonal cell structure as compared to a control (untreated) donor corneal tissue. The subject may have an eye disorder chosen from Fuchs endothelial cell dystrophy or pseudophakic corneal edema. The subject may be a human or non-human mammal, for example.

As mentioned above, according to some aspects of the present disclosure, two or more different compositions comprising a proteoglycan or core protein thereof may be applied to the subject. For example, the composition used to treat the donor corneal tissue may be a first composition, and the method further comprises applying a second composition different from the first composition directly to a surface of a cornea of the eye before and/or after applying the corneal graft, the second composition comprising a proteoglycan or core protein thereof, e.g., decorin proteoglycan or decorin core protein. The second composition may be applied with an applicator having a cavity that contains the second composition, the applicator being in direct contact with the subject's eye.

### BRIEF DESCRIPTION OF THE FIGURES

The accompanying figures, which are incorporated in and constitute a part of this specification, illustrate various examples and together with the description, serve to explain the principles of the present disclosure.

FIG. 1 reports specular microscopy analysis from Example 1.

FIGS. 2A and 2B are micrographs of control samples of human donor cornea, and FIGS. 2C and 2D are micrographs of treated human donor cornea, as discussed in Example 1.

FIG. 3 reports specular microscopy and corenal pachymetry analysis from Example 2.

### DETAILED DESCRIPTION

The singular forms “a,” “an,” and “the” include plural reference unless the context dictates otherwise. The terms “approximately” and “about” refer to being nearly the same as a referenced number or value. As used herein, the terms “approximately” and “about” generally should be understood to encompass ±5% of a specified amount or value. Any aspect described herein as exemplary is not to be construed as preferred or advantageous over other aspects. Rather, the term “exemplary” is used in the sense of example or illustrative. The terms “comprise,” “include,” “have,” “with,” and any variations thereof are used synonymously to denote or describe non-exclusive inclusion.

The present disclosure relates to methods for treating corneal tissue. For example, the present disclosure includes methods for maintaining corneal endothelial cell anatomy, function, and/or structure preceding, during, and/or following a medical procedure. Such medical procedures may include intraocular surgery procedures, including phacoemulsification, high frequency pulsed vacuum cataract surgery procedures, and corneal endothelial transplantation, e.g., Descemet's stripping automated endothelial keratoplasty (DSAEK) and Descemet's membrane endothelial keratoplasty (DMEK), deep anterior lamellar keratoplasty (DALK), anterior lamellar keratoplasty (ALK), lamellar keratoplasty (LK), posterior lamellar keratoplasty (PLK), penetrating keratoplasty (PK), and introduction of an artificial graft.

The methods may include the application of compositions comprising one or more proteoglycans and/or proteoglycan core proteins. Exemplary proteoglycans and core proteins thereof include, for example, decorin, lumican, keratocan, biglycan, lumican, mimican, fibromodulin, and epiphycan. Decorin, lumican, keratocan, biglycan, mimican, fibromodulin, and epiphycan are members of a family of small leucine-rich repeat proteoglycans or SLRPs. The proteoglycan structure includes a core protein with one or more glycosaminoglycan chains. The glycosaminoglycan chains are long, linear carbohydrate polymers of repeating disaccharide (double sugar) units.

Glycosaminoglycans are negatively charged under physiological conditions due to the occurrence of sulfate and uronic acid groups. Decorin is an approximately 100 kDa proteoglycan that includes a 40 kDa core protein and one chondroitin sulfate or dermatan sulfate glycosaminoglycan chain. Biglycan and epiphycan similarly include respective core proteins with chondroitin sulfate or dermatan sulfate glycosaminoglycan. Lumican, keratocan, mimican, and fibromodulin each include the glycosaminoglycan keratan sulfate. SLRPs interact with collagen Type I and II, fibronectin, thrombospondin and TGF_{β}. Animal-derived decorin and recombinant human decorin protein, as well as other SLRPS, are available, e.g., from Sigma Chemical Company and Catalent Pharma Solutions (known as Galacorin™). The source of proteoglycan or core protein thereof may be in solution form or solid form, e.g., as a powder, such as a lyophilized powder.

Decorin and other SLRPs function as a tissue stabilizer and organizer. Decorin is a horseshoe shaped proteoglycan that binds to collagen fibrils in human cornea forming a bidentate ligand attached to two neighboring collagen molecules in the fibril or in adjacent fibrils, helping to stabilize fibrils and orient fibrillogenesis. Decorin appears to be a ubiquitous component of extracellular matrices linking collagen fibrils at binding sites. Other SLRPs perform a similar function. SLRPS regulate fibrillogenesis by interacting with collagen fibrils and extracellular matrix proteins. Corneal transparency is dependent on the size and arrangement of collagen fibrils in the corneal stroma. SLRP binding is understood to play a consequential role in limiting collagen fibril growth and in controlling the arrangement of collagen fibrils to produce transparency. Studies of effects of decorin on in vitro collagen fibrillogenesis have reported inhibition of collagen fibril growth and increase in collagen fibril diameter.

Decorin and other SLRPs also have a number of biological/physiological characteristics, including biological ligand for the EGF receptor and possible regulation of cell growth, down-regulation of TGF_{β}, inhibition of cell attachment and possible anti-adhesion effects, inhibition of cancer cells and inhibition of angiogenesis. Decorin regulates the biology of various types of cancer by modulating the activity of several receptor tyrosine kinases coordinating growth, survival, migration, and angiogenesis. Decorin binds to surface receptors for epidermal growth factor and hepatocyte growth factor with high affinity, and negatively regulates their activity and signaling via robust internalization and eventual degradation. The insulin-like growth factor (IGF)-I system plays a significant role in the regulation of cell growth both in vivo and in vitro. The IGF-I receptor (IGF-IR) is also involved in cellular transformation, owing to its ability to enhance cell proliferation and protect cells from apoptosis.

According to some aspects of the present disclosure, a composition comprising a proteoglycan or core protein thereof is applied to an eye of a subject (e.g., a patient) before, after, and/or during intraocular medical procedure. Such medical procedures include, but are not limited to, cataract surgery, e.g., phacoemulsification and/or high frequency pulsed vacuum cataract procedures. The compositions herein may be incorporated into or added to ophthalmic viscoelastic devices (OVDs) used during a medical procedure, or may constitute an OVD. OVDs include non-crosslinked hyaluronic acid gels that may provide some protection for corneal endothelium during phacoemulsification cataract surgery and/or other intraocular medical procedures.

The compositions and methods herein additionally or alternatively may be used to prepare materials useful in a transplantation procedure. For example, corneal tissue treated according to the methods herein may be used in a medical procedure to supplement and/or replace native corneal tissue. According to some aspects of the present disclosure, the compositions herein may be applied to corneal tissue, such as a donor cornea. Donor corneas may be obtained from an eye bank or other donor repository, for example. The donor cornea may be treated with the compositions herein to render the corneal tissue suitable for implantation in a subject, e.g., a patient. For example, the treated corneal tissue may be used to replace corneal endothelial tissue in a subject having an eye disorder. Exemplary eye disorders that may be treated include, but are not limited to, endothelial disorders such as Fuchs endothelial cell dystrophy and pseudophakic corneal edema.

The compositions herein comprise at least one proteoglycan or core protein thereof. Exemplary proteoglycans suitable for the present disclosure include decorin, biglycan, keratocan, lumican, mimican, fibromodulin, and epiphycan. According to some aspects of the present disclosure, the composition comprises the core protein of decorin, biglycan, keratocan, lumican, mimican, and/or fibromodulin, without any glycosaminoglycan chain. The compositions herein may comprise at least one glycosaminoglycan in addition to and/or as part of the proteoglycan. Such glycosaminoglycans include, e.g., heparin, heparan sulfate, chondroitin sulfate, dermatan sulfate, keratan sulfate, hyaluronic acid, hydroxyproplymethylcellulose or sodium hyaluronate.

The compositions herein may be formulated for application to the eye of a subject (e.g., patient) and/or may be used to treat donor corneal tissue prior to using the donor corneal tissue in a medical procedure. When formulation for application to the eye of a subject, the composition may be a physiologically acceptable formulation. For example, the composition may be formulated for topical application to a subject.

The compositions of the present disclosure may further comprise a pharmaceutically acceptable salt, such as one or more chloride, acetate, and/or citrate salts. Exemplary salts suitable for the present disclosure include, but are not limited to, sodium chloride, potassium chloride, calcium chloride (including calcium chloride dihydate), magnesium chloride (including magnesium chloride hexahydrate), sodium acetate (including sodium acetate trihydrate), and/or sodium citrate (including sodium citrate dihydrate). Additionally or alternatively, the composition may comprise a physiological buffer, such as a phosphate, e.g., monobasic sodium phosphate, disodium hydrogen phosphate and/or potassium dihydrogen phosphate. The composition may comprise one or more other components such as, e.g., antibiotic agents (including, but not limited to, gentamycin and streptomycin), antioxidants (including, but not limited to, gutathione), amino acids, cell nutrients (including, but not limited to, pyruvate), polysaccharides (including, but not limited to, dextran), and/or organic or biological molecules like insulin and ATP

According to some aspects of the present disclosure, the concentration of proteoglycan or core protein thereof in the composition ranges from about 0.001 mg/mL to about 10 mg/mL, such as about 0.001 mg/mL to about 1.0 mg/mL, about 0.005 mg/mL to about 0.1 mg/mL, about 0.01 mg/mL to about 1.0 mg/mL, about 0.05 mg/mL to about 2.0 mg/mL, about 0.07 mg/mL to about 2.5 mg/mL, about 0.1 mg/mL to about 3 mg/mL, about 0.5 mg/mL 1 mg/mL to about 6 mg/mL, such as from about 1 mg/mL to about 5 mg/mL, about 1.5 mg/mL to about 4.5 mg/mL, about 2 mg/mL to about 4 mg/mL, from about 3 mg/mL to about 5 mg/mL, from about 4 mg/mL to about 5 mg/mL, from about 2 mg/mL to about 4 mg/mL, about 0.5 mg/mL to about 2.5 mg/mL, about 1 mg/mL to about 2 mg/mL, or about 3.5 mg/mL to about 5 mg/mL. In some examples, the concentration of proteoglycan may be within a range of about 10 μg/mL to about 500 μg/mL, about 50 μg/mL to about 250 μg/mL, about 100 μg/mL to about 500 μg/mL, about 150 μg/mL to about 300 μg/mL, or about 250 μg/mL to about 500 μg/mL.

The pH of the composition may be balanced. For example, the pH of the composition may range from about 6.0 to about 8.0, such as about 6.5 to about 7.8, about 6.5 to about 7.5, about 6.8 to about 7.6, about 7.0 to about 7.4, about 7.0 to about 7.2, or about 6.8 to about 7.2. The compositions herein may be the form of a solution, e.g., an aqueous solution. According to some aspects of the present disclosure, the composition comprises a balanced salt solution. For example, the composition may comprise one or more of the following salts: sodium chloride, potassium chloride, calcium chloride dehydrate, magnesium chloride hexahydrate, sodium acetate trihydrate, sodium citrate dihydrate, and combinations thereof. The pH of the balanced salt solution may have a pH of about 6.0 to about 8.0, optionally comprising sodium hydroxide and/or hydrochloric acid to adjust pH. The composition may have an osmolality ranging from about 200 mOsm/kg to about 400 mOsm/kg, such as 300 mOsm/kg. In some examples, the composition comprises a buffer solution, e.g., an infusion buffer solution. For example, the composition may comprise phosphate buffer solution, optionally comprising sodium chloride and/or potassium chloride. Other suitable physiological buffers may be used. Exemplary buffer solutions may have a concentration ranging from about 0.005 M to about 1.0 M phosphate buffer, such as from about 0.01 M to about 1.0 M, about 0.01 M to about 0.5 M, about 0.05 M to about 1.0 M, about 0.1 M to about 0.5 M, or about 0.5 M to about 1.0 M. Such compositions may have a concentration of proteoglycan or core protein thereof ranging from about 0.5 mg/mL to about 5 mg/mL, as discussed above.

In at least one example, the composition is formulated for tissue storage. For example, the composition may comprise a preservation medium that includes at least one proteoglycan or core protein thereof. For example, the composition may be used to treat donor corneal tissue, e.g., to preserve endothelial cells prior to introducing the donor corneal tissue into a subject in a medical procedure. Such compositions may comprise, for example, one or more proteoglycans or core proteins thereof (e.g., decorin, biglycan, keratocan, lumican, mimican, and/or fibromodulin, or core protein thereof), optionally in combination with at least one glycosaminoglycan such as, e.g., heparin, heparan sulfate, chondroitin sulfate, dermatan sulfate, keratan sulfate, hyaluronic acid, sodium hyaluronate, or a combination thereof. Additionally or alternatively, the composition may comprise at least one antibiotic, antioxidant, amino acid, cell nutrient, polysaccharide, organic molecule, biologic molecule, or combination thereof.

According to some aspects of the present disclosure, the composition is in the form of a gel, e.g., a viscoelastic gel. For example, the composition may comprise a hyaluronic acid gel that includes one or more proteoglycans or core proteins thereof. In at least one example, the composition comprises decorin core protein or decorin proteoglycan and hyaluronic acid. Such compositions may have a concentration of proteoglycan or core protein thereof ranging from about 0.001 mg/mL to about 10 mg/mL as discussed above. According to some aspects of the present disclosure, the composition in gel form may have a viscosity ranging from about 50,000 mPa sec to about 160,000 mPa sec, such as about 50,000 mPa sec to about 75,000 mPa sec, about 50,000 mPa sec to about 55,000 mPa sec, about 90,000 mPa sec to about 110,000 mPa sec, about 100,000 mPa sec to about 150,000 mPa sec, about 125,000 mPa sec to about 150,000 mPa sec, about 130,000 mPa sec to about 140,000 mPa sec, or about 120,000 mPa sec to about 140,000 mPa sec.

Further for example, the composition may be in the form of a viscoelastic film.

As mentioned above, the source of proteoglycan or core protein thereof may be in solution form or solid form. In either case, the proteoglycan or core protein thereof may be combined with other components of the composition, including water or aqueous solution or gel to obtain the desired concentration. When in solid form, e.g., a powder, for example, the proteoglycan or core protein thereof may be combined with an aqueous solution (e.g., a balanced salt solution or preservation medium) or gel (e.g., hyaluronic acid gel). In some examples, the proteoglycan or core protein thereof may be combined with other components of the composition, and the resulting mixture may form a gel or film after adding the proteoglycan or core protein thereof.

As mentioned above, the composition may be formulated for, and applied to, the eye of a subject (e.g., a patient) before, during, or after an intraocular medical procedure. The subject may be a mammal, such as a human or a non-human mammal (e.g., cat, dog, rabbit, etc.). The composition may be applied to corneal tissue, such as endothelial tissue. In some examples, the composition may be applied to the anterior chamber of the eye. The anterior chamber is the space between the endothelium and the iris, i.e., the fluid-filled space behind the cornea. For example, the proteoglycan(s) or core protein(s) thereof may be added to an infusion buffer solution used to maintain the anterior chamber during an intraocular surgery procedure, such as a phacoemulsification cataract procedure, or added to an OVD. Additionally or alternatively, the proteoglycan(s) or core protein(s) thereof may be added to an infusion solution after removal of an OVD to interact directly with exposed corneal endothelial cells. Before, after, or during the course of an intraocular procedure, the compositions herein may be applied to one or more of the following: the eye, the cornea, endothelial tissue, anterior chamber segment tissue, the posterior chamber of the eye, the retina, and/or the epithelium.

In an exemplary intraocular medical procedure, e.g., cataract surgery, an incision is made at the limbus (the junction of the cornea and sclera) or in the clear cornea. The anterior chamber is filled with viscoelastic gel (optionally a composition of the present disclosure comprising a proteoglycan or core protein thereof), and a circular opening in made in the anterior capsule. The cataract nucleus is subdivided by phacoemulsification using high frequency, ultrasound waves or laser energy. Additionally or alternatively, high frequency pulsed vacuum can also be used to remove the cataract nucleus.

The fragments of the cataract are then removed and then the cortex is flushed from the capsular bag using irrigation/aspiration. The bag is re-inflated with viscoelastic gel (optionally a composition as disclosed herein) and an intraocular lens is placed. The viscoelastic gel is then removed and replaced with a pH balanced solution (optionally a composition of the present disclosure comprising a proteoglycan or core protein thereof), e.g., pH balanced salt solution. The intraocular lens is checked for centration and the wound is checked for leaks. As indicated, one or more compositions as disclosed herein may be, or may be incorporated into, a viscoelastic gel and/or solution and applied to the eye at any point during the medical procedure.

In some examples, the composition directly contacts the native corneal endothelium, which may be exposed during the medical procedure. In at least one example, an infusion solution comprising the proteoglycan(s) or core protein thereof may be applied the anterior chamber as a final step of the intraocular medical procedure or at the completion of the procedure. Exemplary doses of the proteoglycan or core protein thereof per composition volume of the proteoglycan or core protein thereof for treatment during an intraocular procedure may range from about 0.005 mg (5 μg) to about 2.0 mg per total delivery solution or gel volume, e.g., about 0.01 mg to about 2 mg, about 0.05 mg to about 0.5 mg, about 0.1 mg to about 1.0 mg, about 0.5 mg to about 1.8 mg, about 0.75 mg to about 1.5 mg, about 0.5 mg to about 1.25 mg, about 0.75 mg to about 1 mg, about 0.5 mg to about 0.75 mg, about 0.25 mg to about 0.75 mg, about 0.6 mg to about 1.2 mg, about 0.9 mg to about 1.3 mg, or about 1.5 mg to about 1.8 mg per total delivery solution or gel volume. For example, the delivery solution of gel volume may range from about 0.1 mL to about 2.0 mL, such as about 0.1 mL to about 0.5 mL, about 0.2 mL to about 0.35 mL, 0.25 mL to about 0.75 mL, about 0.25 mL to about 0.45 mL, about 0.5 mL to about 1.0 mL, about 0.5 mL to about 2.0 mL, about 1.0 mL to about 2.0 mL, or about 1.0 mL to about 1.5 mL, per application (which may be one or more applications) or per eye (administered in one or more applications). In at least one example, the volume of composition applied is less than 1 mL, e.g., within a range of about from 0.1 mL to about 0.9 mL.

According to some aspects of the present disclosure, the dose of proteoglycan or core protein thereof ranges from about 0.2 mg/100 μL (2 mg/mL) to about 0.6 mg/100 μL (2 mg/mL) per treatment, such as about 0.25 mg/100 μL to about 0.5 mg/100 μL, about 0.25 mg/100 μL to about 0.55 mg/100 μL, about 0.28 mg/100 μL to about 0.475 mg/100 μL, about 0.3 mg/100 μL to about 0.5 mg/100 μL, about 0.35 mg/100 μL to about 0.45 mg/100 μL, about 0.25 mg/100 μL to about 0.5 mg/100 μL, about 0.45 mg/100 μL to about 0.5 mg/100 μL, or about 0.3 mg/100 μL to about 0.4 mg/100 μL. Further exemplary doses range from about 0.005 mg/mL to about 2.0 mg/mL, such as about 0.025 mg/mL to about 1.0 mg/mL, or from about 0.05 mg/mL to about 0.2 mg/mL. The composition may be applied once or multiple times over the course of the intraocular medical procedure.

Endothelial disorders may be treated by supplementing native endothelial cells or replacing a diseased or otherwise damaged endothelial cell layer. For example, disorders such as Fuchs endothelial cell dystrophy or pseudophakic corneal edema may be treated by replacing the endothelial cell layer using a technique called endothelial keratoplasty. In an exemplary procedure, a subject's native endothelial cell layer is supplemented or replaced by a corneal graft. Such methods may include replacing the native diseased endothelial cell layer. For example, the graft may be prepared or precut from tissues obtained from a donor eye bank, e.g., using a micokeratome. In at least one example, the composition may be applied to a cadaver eye or an isolated cornea. In further examples, the composition may be applied to an isolated DMEK graft, an isolated DSAEK graft, an isolated DALK graft, an isolated ALK graft, an isolated LK graft, an isolated PK graft, or an artificial graft.

For effective treatment, the graft should be prepared from donor corneal tissue in which the endothelium is intact and active. The compositions and methods herein are useful to promote the health of donor cornea endothelial cells, including retaining and maximizing endothelial cell numbers and cell density. According to some aspects of the present disclosure, donor corneal tissue stored in a composition comprising a proteoglycan such as a decorin, and may maintain cell density and/or hexagonal cell structure, and/or may exhibit an increase in hexagonal cell structure, as compared to a donor corneal tissue stored in a reference composition without the proteoglycan(s). The composition may comprise one or more proteoglycan(s) in a preservation medium, such as a solution comprising chondroitin sulfate and optionally one or more other components such as dextran and/or an antibiotic agent.

The donor corneal tissue may be treated with the composition for a suitable period of time. For example, the donor corneal tissue may be treated for at least 10 minutes, at least 15 minutes, at least 30 minutes, at least 45 minutes, at least 1 hour, at least 2 hours, at least 4 hours, at least 6 hours, at least 12 hours, at least 18 hours, at least 24 hours, at least 2 days, at least 3 days, at least 5 days, at least 7 days, at least 10 days, or at least 14 days or more. In some examples, the donor corneal tissue is treated for 10 minutes to 1 hour, 30 minutes to 2 hours, 1-6 hours, 8-16 hours, 12-24 hours, 1-30 days, 15-30 days, 1-20 days, 15-20 days, 2-5 days, 7-14 days, 5-15 days, 14-22 days, or 8-12 days. The donor cornea and corneal tissue can be treated before enucleation or in situ removal. The donor corneas and corneal tissue can be treated 10 minutes to 30 days after removal at a temperature of 0.5° C. to 40° C., e.g., a temperature of about 2° C. to about 10° C., about 2° C. to about 8° C., about 12° C. to about 20° C., about 20° C. to about 25° C., about 20° C. to about 30° C., or about 25° C. to about 35° C.

The donor corneal tissue may be formed into a suitable size and shape to serve as a corneal graft before or after treatment. The amount of proteoglycan or core protein thereof suitable for treating donor tissue may be the same as any of the doses described above with respect to application to a subject's eye. For example, the amount of proteoglycan or core protein may from about 0.10 mg to about 2.0 mg per total delivery solution or gel volume (e.g., from about 0.1 mL to about 2 mL). For example, the amount of proteoglycan or core protein thereof may be present in an amount ranging from about 0.5 mg to about 1.8 mg, about 0.75 mg to about 1.5 mg, about 0.5 mg to about 1.25 mg, about 0.75 mg to about 1.0 mg, about 0.5 mg to about 0.75 mg, about 0.25 mg to about 0.75 mg, about 0.6 mg to about 1.2 mg, about 0.9 mg to about 1.3 mg, or about 1.5 mg to about 1.8 mg per total delivery solution or gel volume.

The corneal graft may comprise posterior corneal stroma, e.g., prepared from a donor cornea. Such grafts may have a thickness from about 50 μm to about 700 μm, depending on type of graft. For example, the graft may have a thickness ranging from about 50 μm to about 150 μm, about 100 μm to about 300 μm, about 250 μm to about 400 μm, or about 300 μm to about 350 μm. Exemplary dimensions of the graft may range from about 5 mm to about 12 mm in diameter, such as from about 7 mm to about 10 mm, or from about 8 mm to about 9 mm in diameter. The graft may be prepared from a portion of the anterior cornea. For DSAEK procedures, the graft may comprise endothelium, Descemet's membrane, and at least a portion of corneal stroma. For example, the graft may comprise at least 50 μm of the corneal stroma, such as from about 50 μm to about 150 μm, or from about 75 μm to about 125 μm, e.g., about 100 μm. For DMEK procedures, the graft may comprise or consist of endothelium and Descemet's membrane, without corneal stroma.

According to some examples herein, a medical procedure may include cataract surgery in combination with a keratoplasty procedure, such as Descemet's stripping automated endothelial keratoplasty (DSAEK), Descemet's membrane endothelial keratoplasty (DMEK), deep anterior lamellar keratoplasty (DALK), anterior lamellar keratoplasty (ALK), lamellar keratoplasty (LK), posterior lamellar keratoplasty (PLK), penetrating keratoplasty (PK), or artificial graft. For example, a subject's anterior chamber may be filled with a viscoelastic gel as disclosed herein (e.g., a hyaluronic acid gel comprising one or more proteoglycans such as decorin or decorin core protein), followed by implantation of a graft prepared from donor cornea treated with a composition comprising one or more proteoglycans such as decorin or decorin core protein.

In some examples herein, the composition may be applied directly to the surface of the cornea, e.g., to maintain endothelial cell structure and function. For example, the composition may be applied to the eye before or following an intraocular medical procedure such as cataract surgery and/or endothelial keratoplasty.

Before applying the composition to the cornea surface, a penetration enhancer system may be applied to disrupt the corneal epithelial junctures, e.g., to increase permeability of the epithelium and facilitate delivery of the composition through the epithelium. That is, the penetration enhancer system may permit diffusion of the composition through the epithelial cell layer, Bowman's membrane, and corneal stroma to contact the endothelial cell layer. Suitable penetration enhancers include those described in US 2011/0086802 (referred to therein as “disrupting agents”), incorporated by reference herein. Non-limiting examples of penetration enhancers include anhydrides such as maleic anhydride, succinic anhydride, glutaric anhydride, citractonic anhydride, methyl succinic anhydride, itaconic anhydride, methyl glutaric anhydride, dimethyl glutaric anhydride, and phthalic anhydride; acid chlorides such as oxalyl chloride and malonyl chloride; sulfonyl chlorides such as chlorosulfonylacetyl chloride, chlorosulfonylbenzoic acid, 4-chloro-3-(chlorosulfonyl)-5-nitroebnzoic acid, and 3-(chlorosulfonyl)-P-anisic acid; and sulfonic acids such as 3-sulfobenzoic acid.

An applicator may be used to keep the composition in contact with the eye a sufficient amount of time for the proteoglycan(s) to be absorbed into the cornea. For example, the applicator may include a cup or other cavity to receive a solution as disclosed herein, wherein the applicator is configured to be placed on the eye. The applicator may be configured to tightly cover the corneal surface for effective delivery to the cornea. A suitable anesthetic (e.g., 5% proparicane hydrochloride or the like) may be first applied to avoid patient discomfort. Exemplary applicator devices that may be used include those described in U.S. Pat. Nos. 6,161,544 and 9,399,102, each incorporated by reference herein.

The corneal endothelium is understood to play a role in the maintenance of normal corneal hydration, thickness, and transparency. Application of the compositions herein may help to preserve and maintain cell density, cell hexagonality (i.e., regular endothelial cell structure), and/or to maintain pump function. Cell density and structure (hexagonality) may be determined by specular microscopy. Quantitative specular microscopy may provide a measurement of functionality of endothelial cells associated with the maintenance of corneal hydration and clarity. A suitable specular microscope such as, e.g., a Konan Eyebank KeratoAnalyzer, Konan Cellchek D/D+ may be used to take measurements. In an exemplary test, a fluorescent stain is used to view living cells and delineate cell borders of a sample. For example, the specular microscope may be used to measure one or more of the following parameters to determine a change in endothelial cell characteristics using the compositions and method herein: the distribution of number of cell apices (unit %), the distribution of cell area (unit %), corneal thickness (in the manner of a pachymeter), average cell area (μm²), maximum cell area (μm²), minimum cell area (μm²), number of analyzed cells, cell density (CD), standard deviation of cell area, coefficient of variation (standard deviation of cell area/mean cell area), and/or percent of hexagonal cells.

For example, mean cell density (MCD) and/or mean cell area (MCA) of endothelial cells, as well as variations in cell size (characterized as the coefficient of variation, termed polymegathism), and/or cell shape (characterized as the percent of hexagonal cells, termed pleomorphism), may be measured. MCD is useful to characterize endothelial cell status. Polymegathism and pleomorphism provide an indication of corneal integrity and function. A specular microscope may be used to establish and compare normative data for endothelial characteristics, for example, and investigate the effects of age on endothelial cells. In general, increasing age of a subject may be associated with decreased MCD, increased MCA, increased coefficient of variation in cell size, and decreased percentage of hexagonal cells.

As discussed in Examples 1 and 2 below, the compositions herein when directly applied to the endothelium were found to maintain endothelial mean cell density, exhibit a decrease in the coefficient of variation, and exhibit an increase in cellular hexagonality as compared to a control. These results demonstrate the potential efficacy of proteoglycan solutions in maintaining the corneal endothelium structure and pump function following phacoemulsification cataract surgery and other intraocular surgery procedures. Accordingly, the compositions and methods herein may provide protection to limit and/or prevent endothelial cell destruction during intraocular surgical procedures, including phacoemulsification and high frequency pulsed vacuum cataract surgery. The compositions and methods herein also may help to restore endothelial cell integrity and functionality following intraocular surgery procedures, including phacoemulsification and high frequency pulsed vacuum cataract surgery.

Further aspects of the present disclosure include the following:

1. A method of preparing a corneal graft, the method comprising: treating donor corneal tissue with a composition comprising a proteoglycan or core protein thereof, such as decorin proteoglycan or decorin core protein, wherein the corneal graft includes the treated donor corneal tissue.

2. The method of aspect 1, wherein the composition further comprises heparin, heparan sulfate, chondroitin sulfate, dermatan sulfate, keratan sulfate, hyaluronic acid, or a combination thereof.

3. The method of aspect 1 or 2, wherein the composition further comprises an antibacterial agent.

4. The method of any of aspects 1-3, wherein the composition is a preservation medium.

5. The method of any of aspects 1-4, wherein the donor corneal tissue is treated for at least 10 minutes, e.g., about 10 minutes to about 14 days.

6. The method of any of aspects 1-5, wherein the treated donor corneal tissue has the same cell density as the untreated donor corneal tissue. Include as initially stated.

7. The method of any of aspects 1-6, wherein the treated donor corneal tissue exhibits the same hexagonal cell structure as the untreated donor corneal tissue or exhibits an increase in hexagonal cell structure as compared to the untreated donor corneal tissue.

8. The method of any of aspects 1-7, wherein the donor corneal tissue is obtained from an eye bank.

9. A corneal graft prepared according to the method of any of aspects 1-8.

10. The corneal graft of aspect 9, wherein the corneal graft has a thickness ranging from about 50 μm to about 700 μm.

11. The corneal graft of aspect 9 or 10, wherein the corneal graft has a diameter ranging from about 7 mm to about 10 mm.

12. A method of preparing the corneal graft of any of aspects 9-11.

13. A method of preparing a corneal graft, the method comprising treating donor corneal tissue with a preservation medium comprising a proteoglycan or core protein thereof, such as decorin proteoglycan or decorin core protein; wherein the treated donor corneal tissue exhibits an increase in hexagonal cell structure as compared to the donor corneal tissue; and wherein the corneal graft includes the treated donor corneal tissue.

14. The method of aspect 12 or 13, wherein the donor corneal tissue is treated for at least 10 minutes up to about 30 days, such as about 10 minutes up to about 29 days, or about 10 minutes up to about 14 days.

15. A method of treating a subject, the method comprising applying a corneal graft to an eye of the subject, wherein the corneal graft comprises donor corneal tissue treated with a composition comprising a proteoglycan or core protein thereof, such as decorin proteoglycan or decorin core protein.

16. The method of aspect 15, wherein the subject has an eye disorder chosen from Fuchs endothelial cell dystrophy or pseudophakic corneal edema.

17. The method of aspect 15 or 16, wherein the corneal graft exhibits the same hexagonal cell structure as the untreated donor corneal tissue or exhibits an increased hexagonal cell structure as compared to the untreated donor corneal tissue.

18. The method of any of aspects 15-17, wherein the composition is a first composition, and the method further comprises applying a second composition different from the first composition directly to a surface of a cornea of the eye before and/or after applying the corneal graft, the second composition comprising a proteoglycan or core protein thereof, such as decorin proteoglycan or decorin core protein.

19. The method of aspect 18, wherein the second composition is applied with an applicator having a cavity that contains the second composition, the applicator being in direct contact with the eye.

The following examples are intended to illustrate the present disclosure without, however, being limiting in nature. It is understood that the present disclosure encompasses additional embodiments consistent with the foregoing description and following examples.

### EXAMPLES

### Example 1

The specular microscope has been used to establish and compare normative data for endothelial characteristics in various populations. Investigative studies have shown that with increasing age there is a general trend toward decreased mean cell density, increased mean cell area, increased coefficient of variability in cell size, and decreased percentage of hexagonal cells. Studies were conducted to evaluate the effects of decorin solution on corneal endothelium integrity and structure. In this example, human donor cornea treated with decorin solution were evaluated as compared to human donor cornea stored in a standard preservation medium as a control to investigate effects on preserving and/or increasing the physiology and functionality of endothelial cells in corneal cells.

Intact human donor corneas were used for this study. Donor cornea were processed to obtain the lower layer of the cornea to prepare DSAEK sections containing intact endothelium layers attached corneal stromal tissue. The DSAEK procedures included cutting the human donor cornea with a Moria automated lamellar therapeutic keratoplasty (ALTK) system (Antony, France). The resultant posterior cornea was cut with a 9 mm punch. This donor size was chosen in order to increase the number of donor endothelial cells available to be transplanted, and to ease surgical centration of the donor against a recipient cornea.

One pair of human corneas (as test and control) was used for this evaluation. Paired human corneas were cut by a certified eye bank technician under a validated DSAEK procedure used for preparation of corneas used in human corneal transplantation. The test solution was prepared with 4.7 mg/mL of decorin core protein in neutral pH sodium phosphate buffer containing sodium chloride (pH 7.0±0.2).

The test cornea was treated with 300 μL of decorin solution (1.425 mg decorin) and then placed in Optisol GS (Bausch & Lomb). The paired test and control human corneas were stored at 2-8° C. for 15 days in Optisol GS. Paired human corneas were evaluated at the time of arrival (pre-evaluation) and 15 days post incubation by specular microscopy using a Konan Eyebank KeratoAnalyzer. Three measurements were taken for each control cornea and each test cornea.

The paired test and control corneas were then placed in a serum free intermediate-term corneal preservation medium at 35° C. for 14 additional days to allow for greater endothelial and epithelial cell evaluation. This in vitro corneal model is predictive of endothelial cell viability in vivo post keratoplasty. The samples were then thinned down 1 day in Optisol GS and again evaluated by specular microscopy. Three measurements were again taken for each control cornea and each test cornea.

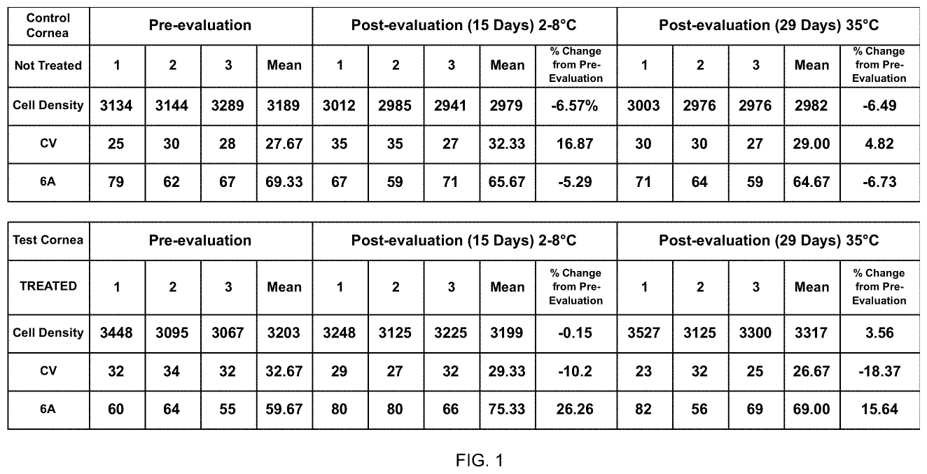

Specular analysis was performed by a built-in validated software program from images of the central cornea marked by the instrument operator. The aim of this quantitative specular microscopy analysis was to assign values to endothelial cells that can provide a measure of their functional status. Three central images were evaluated for both the control and test cornea. Results are provided in FIG. 1 (cell density in cells/mm²; CV=coefficient of variation; 6A=percentage of hexagonal cells and corneal thickness (μm). The percent change of mean cell density, coefficient of variation, percentage of hexagonal cells, and corneal thickness (μm) values are calculated relative to the respective pre-evaluation values.

As shown in FIG. 1, after 15 days storage at 2-8° C. in control medium, Optisol GS, the instrumentation software calculated that the control cornea exhibited a 6.57% decrease in mean cell density (greater loss of cell density), a 16.87% increase in coefficient of variation (increase of variation of cell size), and a 5.29% decrease in hexagonal endothelial cells (decrease in hexagonality cell shape) as compared to the pre-evaluation values. After 15 days at 2-8° C. storage in Optisol GS, the decorin treated test cornea exhibited a 0.15% decrease in mean cell density (greater retention of endothelial cells as compared to control), a 10.2% decrease in coefficient of variation (the treatment reduced the coefficient of variation, or variation in cell size), and a 26.26% increase in hexagonal endothelial cells (the treatment increased the hexagonality of cell shape) as compared to the pre-evaluation values.

As shown in FIG. 1, after an additional 14 days at 35° C./1 day thin down storage in serum-free medium/control medium Optisol GS (29 days total), the instrumentation software calculated that the control cornea exhibited a 6.49% decrease in mean cell density (greater loss of cell density), a 4.82% increase in coefficient of variation (increase of variation of cell size) and a 6.73% decrease in hexagonal endothelial cells (decrease in hexagonality cell shape) as compared to the pre-evaluation values.

As shown in FIG. 1, after an additional 14 days at 35° C./1 day thin down storage in serum-free medium/control medium Optisol GS, (29 days total), the decorin treated cornea exhibited 3.56% increase in mean cell density (greater retention of endothelial cells as compared to control), a 18.37% decrease in coefficient of variation (the treatment reduced the coefficient of variation, or variation in cell size), and a 15.64% increase in hexagonal endothelial cells (the treatment increased the hexagonality of cell shape) as compared to pre-evaluation test cornea values.

These results demonstrate that the decorin treated cornea had improved preservation of endothelial cell density, that is, reduced cell loss in in cell number over time as compared to the untreated control, decreased coefficient of cell variation, and increased percentage of hexagonal endothelial cells as compared to the untreated control.

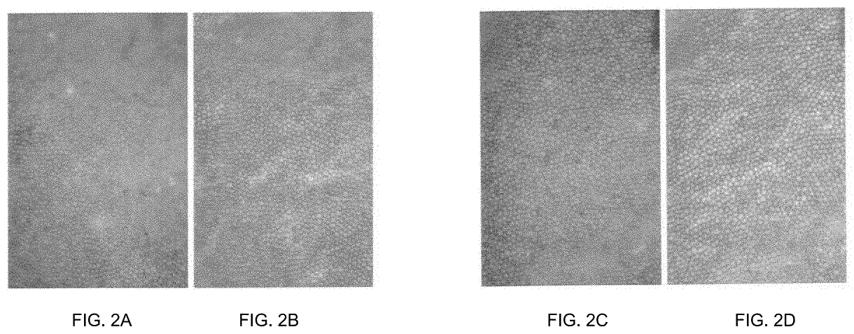

FIGS. 2A-2B show photograph images of the control cornea, and FIGS. 2C-2D show micrograph images of the decorin-treated test corneal after 29 days of storage. Calcein AM fluorescent stain was used to view living cells (see FIGS. 2A and 2C), and alizarin red stain with trypan blue followed by alizarin red S stain was used to visualize cell borders (see FIGS. 2B and 2D). As shown, decorin-treated corneas had viable keratocytes and viable corneal endothelium as visualized by fluorescent calcein staining (FIG. 2C), and uniform central corneal endothelium as visualized after alizarin red S staining (FIG. 2D).

### Example 2

Effects of decorin solutions were evaluated as compared to standard preservation medium as a control to demonstrate effects on preserving and increasing the physiology and functionality of endothelial cells in corneal cells, in the form of human donor cornea.

Studies were conducted to evaluate the effect(s) of decorin treatment on corneal endothelium integrity and structure. The test solution applied to the test cornea was prepared with 4.7 mg/mL of decorin core protein in neutral pH sodium phosphate buffer containing sodium chloride (pH 7.0±0.2). Intact human donor corneas were used for this study.

Paired human donor corneas were evaluated at the time of arrival (pre-evaluation). The test cornea was treated with 300 uL of decorin solution (1.425 mg decorin) and then placed in Optisol GS. The paired test and control human corneas were stored at 2-8° C. for 14 days. The test and control corneas were evaluated at 7 days (for an intermediate evaluation) and 14 days post-incubation by specular microscopy and corneal pachymetry. The test and control corneas were then placed in a serum free intermediate-term corneal preservation medium at 35° C. for 14 additional days and then thinned down and stored for an additional day in Optisol GS, followed by evaluation by specular microscopy, as well as by corneal pachymetry to measure sample thickness.

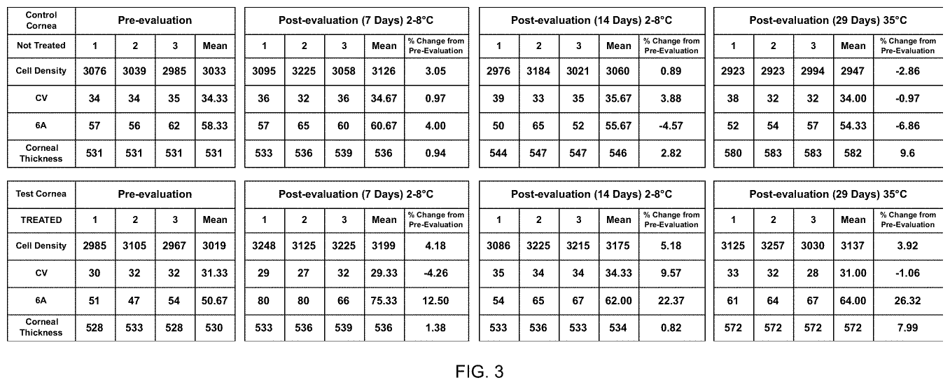

Specular analysis was performed by a built-in validated software program from images of the central cornea marked by the instrument operator. Three central images were evaluated for both the control and test cornea. Results are provided in FIG. 3 (cell density in cells/mm²; CV=coefficient of variation; 6A=percentage of hexagonal cells and corneal thickness (μm). The percent change of mean cell density, coefficient of variation, percentage of hexagonal cells, and corneal thickness (μm) values are calculated relative to the respective pre-evaluation values.

As shown in FIG. 3, after 7 days at 2-8° C. storage in control medium Optisol GS, the control cornea exhibited a 3.05% increase in mean cell density, 0.97% increase in coefficient of variation, 4.00% increase in hexagonal cells, and 0.94% increase in corneal thickness as compared to the pre-evaluation values. After 7 days at 2-8° C. storage in Optisol GS, the decorin treated cornea exhibited a 4.18% increase in mean cell density, a decrease of 4.26% in coefficient of variation, an increase of 12.50% of hexagonal cells and a 1.38% increase in corneal thickness as compared to the pre-evaluation values.

As shown in FIG. 3, after an additional 7 days (total 14 days) at 2-8° C. storage in control medium Optisol GS, the control cornea exhibited a 0.89% increase in mean cell density, a 3.88% increase in coefficient of variation, a 4.57% decrease in hexagonal cells, and a 2.82% increase in corneal thickness as compared to the pre-evaluation values. After an additional 7 days (total 14 days) at 2-8° C. storage in Optisol GS, the decorin treated cornea exhibited a 5.18% increase in mean cell density, an increase of 9.57% in coefficient of variation, an increase of 22.37% of hexagonal cells, and a 0.82% increase in corneal thickness as compared to the pre-evaluation values.

As shown in FIG. 3, after an additional 14 days at 35° C./1 day thin down storage in serum-free medium/control medium Optisol GS (29 days total), the control cornea exhibited a 2.86% decrease in mean cell density, a decrease of 0.97% in coefficient of variation, a decrease of 6.86% of hexagonal cells, and a 9.60% increase in corneal thickness as compared to the pre-evaluation values.

As shown in FIG. 3, after an additional 14 days at 35° C./1 day thin down storage in serum-free medium/control medium Optisol GS, (29 days total), the decorin treated cornea exhibited a 3.92% increase in mean cell density, a decrease of 1.06% in coefficient of variation, an increase of 26.32% of hexagonal cells, and a 7.99% increase in corneal thickness as compared to the pre-evaluation values.

After the total of 29 days of the study, the control and test corneas both had viable multilayer corneal epithelium, viable keratocytes, and viable corneal endothelium as visualized by intensely fluorescent calcein staining. Additionally, both the control and test samples had uniform central corneal endothelial cells seen after Alizarin Red S staining.

These results demonstrate that the decorin-treated test cornea had improved preservation of endothelial cell density, that is, reduced cell loss in in cell number over time as compared to the untreated control, decreased coefficient of variation (decrease variation of cell size), and increased hexagonal structure (increase in hexagonality cell shape) as compared to the untreated control.

### Example 3

Additional studies are conducted using intact donor corneas. The studies are similar to those described in Example 1, but using decorin proteoglycan in a preservation medium instead of decorin core protein in buffer solution. Three sets of paired human corneas are used for this evaluation. Test corneas are treated with Optisol GS corneal preservation medium containing 500 μg/mL of decorin proteoglycan (Bovine-derived, Sigma Aldrich); control corneas are stored in Optisol GS corneal preservation medium. Paired corneas are initially evaluated by specular microscopy before treatment and then at 14 days after storage at 35° C. After 14 days at 35° C., the control corneas exhibit a decrease in mean cell density and a decrease in hexagonal cells. In comparison, the decorin proteoglycan treated cornea exhibit an increase in mean cell density and an increase in hexagonal cells compared to the control corneas. After 29 days of storage, decorin proteoglycan treated corneas have viable keratocytes and viable corneal endothelium as visualized by fluorescent calcein staining and uniform central corneal endothelium as visualized after Alizarin Red S staining.

### Example 4

Additional studies are conducted using intact human donor corneas to test treatment with a proteoglycan in a hyaluronic acid gel. Two sets of paired human corneas are used for this evaluation. Test corneas are treated with 1 mL of an aqueous solution comprising hyaluronic acid (16 mg/mL) and decorin proteoglycan or decorin core protein (2 mg/mL) in sodium chloride phosphate buffer (pH 6.8-7.6); control cornea are treated in a balanced aqueous salt solution (pH 7.0±0.2). Paired corneas are initially evaluated by specular microscopy before treatment and then at 14 days after storage in balanced salt solution at 35° C. After 14 days at 35° C., the control corneas exhibit a decrease in mean cell density and a decrease in hexagonal in endothelial cells. In comparison, the decorin treated cornea exhibit maintenance of mean cell density and an increase in hexagonal cells. After 29 days of storage, decorin treated corneas have viable keratocytes and viable corneal endothelium as visualized by fluorescent calcein staining and uniform central corneal endothelium as visualized after alizarin red S staining.

### Example 5

A cataract procedure is performed on a human subject as follows. An incision is made at the limbus into the anterior chamber using a metal or diamond blade, or a femtosecond laser. A 30 gauge cannula is inserted and about 1 mL of an OVD, a viscoelastic gel comprising hyaluronic acid and decorin proteoglycan or decorin core protein (1-2 mg) is introduced into the anterior chamber so as to completely fill the anterior chamber. The base hyaluronic acid gel also comprises sodium chloride phosphate buffer (pH 6.8-7.6) and has a viscosity of about 123,000 cP. Cataracts are disrupted and removed via phacoemulsification. Following cataract removal, the OVD is removed by aspiration and replaced with a physiological solution. Pre- and post-operative examination of the endothelium is conducted using specular microscopy and corneal thickness is measured by ultrasonic pachymetry. Results show preservation of endothelial structure and maintenance of normal corneal thickness following the cataract removal procedure.

Although the present disclosure includes reference to exemplary embodiments, the disclosure is not limited thereto. It is understood that a person of ordinary skill in the art will ascertain additional embodiments according to the principles described herein and without departing from the spirit and scope thereof, and can make various changes and modifications of the invention to adapt it to various usages and conditions. Those skilled in the art will recognize or be able to ascertain using no more than routine experimentation, many equivalents to the exemplary embodiments herein. Such equivalents are intended to be encompassed in the scope of the present disclosure.

1. A method of treating a subject to preserve and maintain endothelial cell density of the subject, the method comprising:
injecting a composition into an anterior chamber of an eye of the subject during an intraocular medical procedure, wherein the composition comprises a proteoglycan or proteoglycan core protein, and a pharmaceutically-acceptable salt, and wherein the composition is applied to endothelial tissue, thereby preserving and maintaining endothelial cell density.

2. The method of claim 1, wherein the proteoglycan or proteoglycan core protein comprises decorin, biglycan, keratocan, lumican, mimican, fibromodulin, epiphycan, or a core protein thereof.

3. The method of claim 1, wherein the composition comprises an aqueous solution or a viscoelastic gel.

4. The method of claim 1, wherein a concentration of the proteoglycan or proteoglycan core protein in the composition ranges from about 0.001 mg/mL to about 10 mg/mL

5. The method of claim 1, wherein the salt comprises sodium chloride, potassium chloride, calcium chloride, magnesium chloride, sodium acetate, sodium citrate, or a combination thereof.

6. The method of claim 1, wherein the composition further comprises hyaluronic acid, sodium hyaluronate, chondroitin sulfate, dermatan sulfate, heparin sulfate, keratin sulfate, hydroxylpropylmethylcellulose, recombinant human collagen, or a combination thereof.

7. The method of claim 1, wherein a pH of the composition ranges from 6.0 to 8.0.

8. The method of claim 1, wherein the subject is a human or a non-human mammal.

9. (canceled)

10. The method of claim 1, wherein the intraocular medical procedure is cataract surgery.

11. The method of claim 10, wherein the cataract surgery is phacoemulsification cataract surgery or high frequency pulsed vacuum cataract surgery.

12. The method of claim 1, wherein a total volume of the composition injected into the anterior chamber ranges from about 0.050 mL to about 2.0 m L.

13. (canceled)

14. The method of claim 1, wherein the composition is a first composition, and the method further comprises applying a second composition different from the first composition directly to a surface of a cornea of the eye before or after performing the intraocular medical procedure, the second composition comprising decorin proteoglycan or decorin core protein.

15. (canceled)

16. A method of treating a subject to preserve and maintain endothelial cell density of the subject, the method comprising:
applying a composition to endothelial tissue of the subject during cataract surgery, wherein the composition directly contacts a native corneal endothelium of the eye, wherein the composition comprises a proteoglycan or proteoglycan core protein, and wherein the composition preserves and maintains endothelial cell density, the composition being in the form of an aqueous solution, a viscoelastic gel, or a viscoelastic film.

17. The method of claim 16, wherein the proteoglycan or proteoglycan core protein is decorin proteoglycan or decorin core protein, and the composition further comprises hyaluronic acid, sodium hyaluronate, chondroitin sulfate, dermatin sulfate, heparin sulfate, keratin sulfate, hydroxylpropylmethylcellulose, recombinant human collagen, or a combination thereof.

18. The method of claim 16, wherein the cataract surgery includes phacoemulsification cataract surgery or high frequency pulsed vacuum cataract surgery.

19. A method of treating a subject to preserve and maintain endothelial cell density of the subject, the method comprising:
applying a composition comprising decorin proteoglycan or decorin core protein to an eye of the subject during an intraocular medical procedure that includes a phacoemulsification process;
wherein the composition is an aqueous solution, viscoelastic gel and/or film;
wherein the composition directly contacts a native corneal endothelium of the eye, a concentration of the decorin proteoglycan or decorin core protein in the composition ranging from about 0.1 mg/mL to about 3 mg/mL; and
wherein the intraocular medical procedure treats cataracts, and the composition is applied before, during, or after the phacoemulsification process, thereby preserving and maintaining endothelial cell density.

20. The method of claim 19, wherein a total volume of the composition ranges from about 0.050 mL to about 2.0 mL.

21. (canceled)

22. The method of claim 19, wherein the composition is injected into an anterior chamber of the eye.
